(19) United States
(12) Patent Application Publication
(10) Pub. No.: US 2005/0118596 A1
(43) Pub. Date: Jun. 2, 2005

(54) Method for screening for compounds having hdac inhibitory activity

(76) Inventors: Fredericus Alphonsus Maria Asselbergs, Riehen (CH); Peter Wisdom Atadja, Action, MA (US); Jonathan Hall, Dornach (CH); Bernd Kinzel, Allschwill (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/503,186

(22) PCT Filed: Feb. 7, 2003
(86) PCT No.: PCT/EP03/01228

(30) Foreign Application Priority Data
Feb. 8, 2002 (US) 60355311

(51) Int. Cl. C12Q 1/68; G01N 33/574

(52) U.S. Cl. 435/6; 435/7.23

(57) ABSTRACT

The invention relates to the use of Fra-1 levels as a marker for HDAC inhibition. Also disclosed are in vivo and in vitro methods for screening a compound for HDAC inhibitory activity, as well as methods for monitoring the therapeutic efficacy of an HDAC inhibitor in a subject in vivo and for determining resistance to an HDAC inhibitor in vitro or in vivo.

### BACKGROUND OF THE INVENTION

Histone deacetylases (HDACS) are enzymes that are important regulators of chromatin structure and transcription that modulate the cell cycle, hormone signalling and development. Numerous anti-proliferative effects have been reported for agents that inhibit histone deacetylation, for example, inhibitors of histone deacetylase such as phenylbutyrate and trichostatin A have shown promise in the treatment of promyelocytic leukemia. In addition, butyrate decreases the expression of pro-inflammatory cytokines TNF-α, TNF-β, IL-6, and IL1-β most likely through inhibition of NFkB activation and inhibition of histone deacetylases. Also, trapoxin A (Tpx A), a microbially derived cyclotetrapeptide (Itazaki et al. J. Antibiot. 43(12):1524-1532 (1990)) has been shown to bind to and potently inhibit histone deacetylase 1 (HDAC1) (Tauton et al. Science 272:408-411 (1996)).

We have now surprisingly discovered that mRNA for Fra-1, a member of the Fos family of proteins implicated in the regulation of cell growth, is down regulated in cells exposed to Tpx A and other HDAC inhibitors. Thus, message levels of Fra-1 can be used as a marker for HDAC activity. The invention provides methods for screening for HDAC inhibition and HDAC inhibitors in vitro and in vivo by detecting Fra-1 levels. The invention also relates to a method for monitoring the therapeutic efficacy of an HDAC inhibitor in a subject in vivo as well as in vivo and in vitro methods to determine resistance to an HDAC inhibitor.

### SUMMARY OF THE INVENTION

In one aspect, the invention pertains to a method for screening for HDAC inhibition in cells in vitro comprising a). detecting Fra-1 levels in said cells and in control cells and b). comparing Fra-1 levels in said cells and in said control cells wherein HDAC inhibition is associated with Fra-1 level down regulation and wherein similar or up regulated levels of Fra-1 compared to control levels indicates that HDACs are not inhibited in said cells in vitro.

In another aspect, the invention pertains to a method for screening for HDAC inhibition in a subject in vivo comprising a). detecting Fra-1 levels in said subject in vivo and in a control subject; and b). comparing Fra-1 levels in said subject in vivo and in said control subject wherein HDAC inhibition is associated with Fra-1 level down regulation and wherein similar or up regulated levels of Fra-1 compared to control levels indicates that HDACs are not inhibited in said subject in vivo.

In another aspect the invention pertains to a method for screening a compound for HDAC inhibitory activity in vitro, comprising a). administering a compound to cells in vitro; b). assaying for Fra-1 levels in said cells and in control cells to which no compound has been administered; and c) comparing levels of Fra-1 in said cells and in said control cells wherein HDAC inhibition is associated with Fra-1 level down regulation and wherein similar or up regulated levels of Fra-1 in said cells compared to Fra-1 levels in the control cells indicate that said compound does not have HDAC inhibitory activity.

In another aspect the invention pertains to a method for screening a compound for HDAC inhibitory activity in a subject in vivo, comprising a). assaying for Fra-1 levels in said subject; b). administering a compound to said subject; c). reassaying for Fra-1 levels in said subject after administration of the compound; and d) comparing Fra-1 levels in said subject before and after administration of the compound wherein HDAC inhibition is associated with Fra-1 level down regulation and wherein similar or up regulated levels of Fra-1 in said subject after administration of the compound compared to levels before compound administration indicate that said compound does not have HDAC inhibitory activity.

In yet another aspect, the invention pertains to a method for monitoring the therapeutic efficacy of a known HDAC inhibitor In a subject comprising a). detecting Fra-1 levels in said subject before and after treatment with said HDAC inhibitor; and b). comparing Fra-1 levels in said subject wherein HDAC inhibition is associated with Fra-1 level down regulation and wherein down regulation of Fra-1 levels in said subject after treatment compared to levels before treatment indicate that the HDAC inhibitor is therapeutically effective.

In still another aspect, the invention relates to a method to determine resistance of a cell to a known HDAC inhibitor comprising a). administering said HDAC inhibitor to cells in vitro; b). screening for Fra-1 levels in said cells and in control cells to which no HDAC inhibitor has been administered, and c). comparing levels of Fra-1 in said cells and in said control cells wherein HDAC inhibition is associated with Fra-1 level down regulation and wherein a lack of Fra-1 level down regulation in said cells compared to Fra-1 levels in the control cells indicates resistance of said cells to the HDAC inhibitor.

In another aspect, the invention relates to a method to determine resistance of a subject to a known HDAC inhibitor comprising a). detecting Fra-1 levels in said subject before and after treatment with said HDAC inhibitor; and b). comparing Fra-1 levels in said subject wherein HDAC inhibition is associated with Fra-1 level down regulation and wherein a lack of Fra-1 level down regulation in said subject after treatment compared to Fra-1 levels before treatment indicates resistance of said subject to the HDAC inhibitor.

In a related aspect the invention relates to a method of diagnosing a proliferative disease susceptible to treatment with HDAC inhibitory agents, which comprises measuring in cells of the subject which exhibits the proliferative disease a decreased level of Fra-1 mRNA or Fra-1 protein. The method preferably takes place ex vivo.

In another related aspect Fra-1 is used as a biomarker for HDAC inhibitory activity.

In related embodiments of the methods discussed above, Fra-1 protein levels and/or mRNA levels, may be assayed.

### DETAILED DESCRIPTION OF THE INVENTION

It has been found that the message level of Fra-1 is down regulated in mammalian cells exposed to HDAC inhibitors. Because Fra-1 message is consistently down regulated regardless of class of HDAC inhibitor tested, these data indicate that lowered message levels of this protein can be used as a marker for HDAC inhibition in a biological system.

While Fra-1 down regulation does not necessarily indicate HDAC inhibition in a cell culture or in vivo subject, data disclosed herein indicate that HDAC inhibition is associated with Fra-1 message level down regulation. Based on this observation, it is clear that a normal or up regulated level of Fra-1 in a culture or subject compared to control levels definitively indicates that HDACs are not inhibited in said culture or subject. Such information can be used to biochemically characterize a particular cell type, including, for example, primary cultures of diseased cells from a subject or established cell lines as well as provide information regarding a disease state or other pathological condition in vivo and may thus provide useful information regarding appropriate clinical treatment options.

In one aspect, therefore, the present invention pertains to a method for screening for HDAC inhibition in vitro or in vivo comprising detecting Fra-1 levels in vitro or in vivo and comparing with Fra-1 levels in appropriate controls. In this case, “appropriate controls” refers to cultures or in vivo subjects (as the case may be) which may be used to provide Fra-1 levels for comparison and will be familiar to one of skill in the art. Similarly, conventional scientific techniques and procedures familiar to one of skill in the art may be employed to practice this screening method.

HDAC inhibitors may be suitable for use as therapeutic agents in mammals, including animals in veterinary medicine or humans, in need of treatment of diseases in which HDAC inhibition is desirable. Such conditions include, but are not limited to, atherosclerosis, inflammatory bowel disease, host inflammatory or immune response, psoriasis and conditions associated with abnormal cell proliferation, such as cancer. In a preferred embodiment the condition is a proliferative disease such as cancer. Thus, given the clinical importance of HDAC inhibitors, a method to facilitate the detection of such useful therapeutic compounds from a chemical compound library of thousands is of significant value. Based on the surprising discovery that HDAC inhibition is associated with Fra-1 message down regulation, it is contemplated herein that Fra-1 message levels can be used in a method to facilitate the identification of novel HDAC inhibitors by permitting the identification of compounds that are clearly not HDAC inhibitors. Compounds which are not HDAC inhibitors can be identified as those compounds which do not cause a down regulation in Fra-1 levels (e.g. mRNA levels and/or protein levels) compared to appropriate controls. These compounds may then be eliminated from the “list” of possible HDAC inhibitors and need not be tested further. Attention may then be focused on those compounds that actually cause down regulation of Fra-1 levels which may then be further assayed using conventional methods to better characterize effects on HDAC activity.

Thus, in another aspect, the invention pertains to a method for screening a compound for HDAC inhibitory activity in vivo comprising assaying for Fra-1 levels in a subject, administering a compound to said subject; reassaying for Fra-1 levels in said subject after administration of the compound; and comparing Fra-1 levels in said subject before and after administration of the compound wherein HDAC inhibition is associated with Fra-1 level down regulation and wherein similar or up regulated levels of Fra-1 in said subject after administration of the compound compared to levels before compound administration indicates that said compound does not have HDAC inhibitory activity.

The in vivo screening assay may be performed using conventional methods. For example, conventional methods may be used to assay levels of Fra-1 mRNA in vivo in a biological sample taken from a subject before and after compound administration. Similarly, test subjects may include, but are not limited to, conventional experimental animal models such as mouse xenograft, orthotopic or metastatic tumor models as well as human patients in controlled, clinical studies familiar to one of skill in the art.

The screening method may also be performed in vitro. Thus, in another aspect the invention pertains to a method for screening a compound for HDAC inhibitory activity in vitro, comprising administering a compound to cells in vitro, assaying for Fra-1 levels in said cells and in control cells to which no compound has been administered, and comparing levels of Fra-1 in said cells and in said control cells wherein HDAC inhibition Is associated with Fra-1 level down regulation and wherein similar or up regulated levels of Fra-1 in said cells compared to Fra-1 levels in control cells indicate that said compound does not have HDAC inhibitory activity.

Like the in vivo method, the in vitro screening method may be performed using techniques familiar to one of skill in the art. For example, a compound may be screened in vitro using primary cells isolated from human or other mammalian subjects or using a variety of cell lines such as H1299, A549, HCT116 or any other cells in which Fra1 mRNA may be detectably expressed. As used herein, “cells in which Fra-1 levels may be detectably expressed” refers to cells which express levels of Fra-1 mRNA and/or Fra-1 protein such that said levels are sufficient for detection according to conventional methods. Such cell lines are available commercially, for example, from the ATCC (Manassas, Va.) and may be discerned by one of skill in the art without undue experimentation. Likewise, a test compound may be administered to cells in vitro and down regulation of Fra-1 assayed according to conventional methods.

The present invention also provides a method for monitoring the therapeutic efficacy of a known HDAC inhibitor in a subject comprising detecting Fra-1 levels in a said subject before and after treatment with said HDAC inhibitor; and comparing Fra-1 levels in said subject wherein HDAC inhibiton is associated with Fra-1 level down regulation and wherein down regulation of Fra-1 levels in said subject after treatment compared to levels before treatment indicate that the HDAC inhibitor is therapeutically effective. For example, circulating tumor epithelial cells, may be purified from the blood of patients undergoing HDAC inhibitor treatment (i.e. biological samples obtained before and after treatment), RNA purified from these cells and Fra-1 levels determined in the samples by RT-PCR according to conventional methods. A lack of Fra-1 down regulation in a patient to whom an HDAC inhibitor has been administered would be indicative of a lack of HDAC inhibition in said patient and would therefore indicate a corresponding lack in therapeutic efficacy.

Thus, Fra-1 levels may be used as a clinical marker to optimize the dosage and the regimen of an HDAC inhibitor by monitoring Fra-1 levels in the subject's biological sample and dosing to achieve a desirable level of down regulation. Accordingly, the method of the present invention can be used to monitor the therapeutic efficacy of a compound and/or to find a therapeutically effective amount or regimen for the selected compound, thereby individually selecting and optimizing a therapy for a patient. Factors for consideration in this context include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an HDAC inhibitor to be administered will be governed by such considerations, and is the minimum amount necessary to prevent, ameliorate, or treat the disease. Such amount is preferably below the amount that is toxic to the host or renders the host significantly more susceptible to infections.

Levels of expression of Fra-1 can be assayed from a biological sample by any known method, including conventional techniques of RNA quantitation such as Northern blot analysis, quantitative PCR or DNA microarrays (e.g. as commercialised by Affymetrix, Santa Clara, Calif.). As used herein, “biological sample” may comprise blood, urine or other biological material which may be used to assay Fra-1 mRNA or protein levels.

As data indicate that HDAC inhibition is associated with Fra-1 down regulation, one may analyze Fra-1 levels in the presence of an HDAC inhibitor as a way to detect the effect of an HDAC inhibitor in vitro or in vivo.

In a another aspect the invention relates to a method of diagnosing a proliferative disease susceptible to treatment with HDAC inhibitory agents, which comprises measuring in cells of the subject which exhibits the proliferative disease a decreased level of Fra-1 mRNA or Fra-1 protein. Said measuring preferably takes place ex vivo, i.e. outside of the body, for instance using tissue or blood which had previously been isolated from said subject.

Specifically, in another aspect, the invention relates to a method to determine resistance of a cell to a known HDAC inhibitor comprising administering said HDAC inhibitor to cells in vitro, screening for Fra-1 levels in said cells and in control cells to which no HDAC inhibitor has been administered, and comparing levels of Fra-1 in said cells and in said control cells wherein HDAC inhibition is associated with Fra-1 level down regulation and wherein a lack of Fra-1 level down regulation in said cells compared to Fra-1 levels in the control cells indicates resistance of said cells to the HDAC inhibitor. Cells that may be analyzed according to this method include, but are not limited to, tumor cell lines as well as primary cultures of neoplastic or other cells obtained from a patient's biological sample.

Similarly, in a related aspect, the invention relates to a method to determine resistance of a subject to a known HDAC inhibitor comprising a). detecting Fra-1 levels in said subject before and after treatment with said HDAC inhibitor; and b). comparing Fra-1 levels in said subject wherein HDAC inhibition is associated with Fra-1 level down regulation and wherein a lack of Fra-1 level down regulation in said subject after treatment compared to Fra-1 levels before treatment indicates resistance of said subject to the HDAC inhibitor. As is the case with all aspects of the invention disclosed herein, administration of HDAC inhibitors and analysis of Fra-1 levels in vitro and in vivo in order to determine resistance to an HDAC inhibitor may be performed according to a variety of conventional methods familiar to one of skill in the art.

In addition to measuring mRNA levels of Fra-1 as discussed in the various aspects above, it is also contemplated herein that one may also analyze Fra-1 protein levels in such cases where HDAC inhibition is not only associated with down regulation of Fra-1 mRNA levels but also with a corresponding down regulation in Fra-1 protein levels in vivo or in vitro. Levels of Fra-1 protein may be detected using conventional techniques, for example, immunoassays or electrophoresis assays. For example, immunoassays can be used to detect or monitor levels of Fra-1 in a biological sample using Fra-1 specific polyclonal or monoclonal antibodies in any standard immunoassay format to measure Fra-1. ELISA (enzyme linked immunosorbent assay) type assays as well as conventional Western blotting assays using monoclonal antibodies are also exemplary assays that can be utilized to make direct determination of levels of the marker protein. Antibodies specific to Fra-1 are available commercially (for example, rabbit anti-human Fra-1 polyclonal antibody (Santa Cruz Biotechnology, Santa Cruz, Calif., USA) or can be produced in accordance with known methods. For example, monoclonal antibodies against Fra-1 may be produced according to conventional methods, for example, as described in Harlow, E. and Lane, eds., 1988, “Antibodies: A Laboratory Manual”, Cold Spring Harbor Laboratory Press, Cold Spring Harbor.

In practicing the present invention, many other conventional techniques in molecular biology may be used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The following examples further illustrate the present invention and are not intended to limit the invention.

### EXAMPLES

### Example 1

### Differential Expression of Fra-1 in H1299 and A549 Cells After TPX Treatment

Materials and Methods

Various compounds that inhibit or modulate the enzymatic activities of HDACs (HDAC inhibitors) include Tpx A, phenylbutyrate, trichostatin A as well as the benzamide known as MS-27-275. (Jung (2001) Current Medicinal Chemistry 8, 1505-1511). Data disclosed herein are the results of experiments using Tpx A (prepared according to the method disclosed in Itazaki et al., J. Antibiotics (Tokyo) 43(12):1524-32 (1990)) and also using several hydroxamate compounds which are histone deacetylase inhibitors, N-Hydroxy-3-[4-[[[2-(1H-indol-3-yl)-ethyl]-amino]methyl]phenyl]-2E-2-propenamide, referred to herein as “Compound A” and N-Hydroxy-3-[4-[[(2-hydroxyethyl)[2-(1H-indol-3-yl)-ethyl]-amino]methyl]phenyl]-2E-2-propenamide, referred to herein as “Compound B”. These compounds and their synthesis are discussed in detail in WO02/22577.

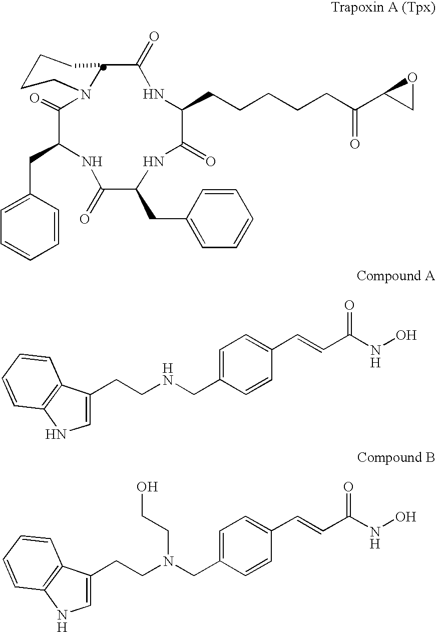

Compound A may be prepared according to the following synthesis: 4-formylcinnamic acid methylester is produced by adding 4-formylcinnamic acid (25 g, 0.143 mol) in MeOH and HCl (6.7 g, 0.18 mol). The resulting suspension is heated to reflux for 3 hours, cooled and evaporated to dryness. The resulting yellow solid is dissolved in EtOAc, the solution washed with saturated NaHCO₃, dried (MgSO₄) and evaporated to give a pale yellow solid which is used without further purification (25.0 g, 92%). To a solution of tryptamine (16.3 g, 100 mmol) and 4-formylcinnamic acid methylester (19 g, 100 mmol) in dichloroethane, NaBH(OAc)₃ (21 g, 100 mmol) is added. After 4 hours the mixture is diluted with 10% K₂CO₃ solution, the organic phase separated and the aqueous solution extracted with CH₂Cl₂. The combined organic extracts are dried (Na₂SO₄), evaporated and the residue purified by flash chromatography to produce 3-(4-{[2-(1H-indol-3-yl)-ethylamino]-methyl}-phenyl)-(2E)-2-propenoic acid methyl ester (29 g). A solution of KOH (12.9 g 87%, 0.2 mol) in MeOH (100 mL) is added to a solution of HONH₂.HCl (13.9 g, 0.2 mol) in MeOH (200 mL) and a precipitate results. After 15 minutes the mixture is filtered, the filter cake washed with MeOH and the filtrate evaporated under vacuum to approximately 75 mL. The mixture is filtered and the volume adjusted to 100 mL with MeOH. The resulting solution 2M HONH₂ is stored under N₂ at −20° C. for up to 2 weeks. Then 3-(4-{[2-(1H-indol-3-yl)-ethylamino]-methyl}-phenyl)-(2E)-2-propenoic acid methyl ester (2.20 g, 6.50 mmol) is added to 2 M HONH₂ in MeOH (30 mL, 60 mmol) followed by a solution of KOH (420 mg, 6.5 mmol) in MeOH (5 mL). After 2 hours dry ice is added to the reaction and the mixture is evaporated to dryness. The residue is dissolved in hot MeOH (20 mL), cooled and stored at −20° C. overnight. The resulting suspension is filtered, the solids washed with ice cold MeOH and dried under vacuum, producing N-Hydroxy-3-[4-[[[2-(1H-indol-3-yl)-ethyl]-amino]methyl]phenyl]-2E-2-propenamide (m/z 336 [MH⁺]).

Compound B may be prepared according to the following synthesis: A solution of 3-(4-{[2-(1H-indol-3-yl)-ethylamino]-methyl}-phenyl)-(2E)-2-propenoic acid methyl ester (12.6 g, 37.7 mmol), (2-bromoethoxy)-tert-butyldimethylsilane (12.8 g, 53.6 mmol), (i-Pr)₂NEt, (7.42 g, 57.4 mmol) in DMSO (100 mL) is heated to 50° C. After 8 hours the mixture is partitioned with CH₂Cl₂/H₂O. The organic layer is dried (Na₂SO₄) and evaporated. The residue is chromatographed on silica gel to produce 3-[4-({[2-(tert-butyldimethylsilanyloxy)-ethyl]-[2-(1H-indol-3-yl)-ethyl]-amino}-methyl)-phenyl]-(2E)-2-propenoic acid methyl ester (13.1 g). Following the procedure described above for the preparation of Compound A, 3-[4-({[2-(tert-butyidimethylsilanyloxy)-ethyl]-[2-(1H-indol-3-yl)-ethyl]-amino}-methyl)-phenyl]-(2E)-2-propenoic acid methyl ester (5.4 g, 11 mmol) is converted to N-hydroxy-3-[4-({[2-(tert-butyidimethylsilanyloxy)-ethyl]-[2-(1H-indol-3-yl)-ethyl]-amino)-methyl)-phenyl]-(2E)-2-propenamide (5.1 g,) and used without further purification. The hydroxamic acid (5.0 g, 13.3 mmol) is then dissolved in 95% TFA/H₂O (59 mL) and heated to 40-50° C. for 4 hours. The mixture is evaporated and the residue purified by reverse phase HPLC to produce N-Hydroxy-3-[4-[[(2-hydroxyethyl)[2-(1H-indol-3-yl)-ethyl]-amino]methyl]phenyl]-2E-2-propenamide as the trifluoroacetate salt (2.19 g).

Human H1299 non-small cell lung carcinoma cells (ATCC, Manassas, Va.) (ATCC #CRL-5803) are cultured in RPMI 1640 medium (Life Technologies, Gaithersburg, Md.) containing 10% fetal bovine serum (FBS, Life Technologies) plus 50 μg/ml Gentamycin (Life Technologies) in a humidified incubator with a 5% CO₂ atmosphere at 37° C. Prior to Tpx A treatment, H1299 cells are plated at a density of 2×10⁵ cells/well in 6 well plates (Costar, Corning, N.Y.) and grown up for 24 h. Tpx A (1 mM stock solution in DMSO) is added to the cells at concentrations from 1 to 1000 nM and cells are incubated for either 24 or 48 hours at 37° C.

Human A549 non-small lung carcinoma cells (ATCC CCL-185, Manassas, Va.) are cultured in DMEM medium (Life Technologies, Gaithersburg, Md.) containing 4500 mg/l glucose, 10% fetal bovine serum (FBS, Life Technologies) plus 50 μg/ml Gentamycin (Life Technologies) in a humidified incubator with a 5% CO₂ atmosphere at 37° C. Prior to Tpx A treatment, A549 cells are plated at a density of 2×10⁵ cells/well in 6 well plates (Costar, Corning, N.Y.) and grown up for 24 h. Tpx A (1 mM stock solution in DMSO) is added to the cells at concentrations from 1 to 1000 nM and cells are incubated for either 24 or 48 hours h at 37° C.

Total RNA from Tpx A treated as well as untreated H1299 and A549 cells is prepared using the RNeasy Mini kit (Qiagen, Hilden, Germany) according to the manufacturers instructions. The RNA is recovered in 50 μl DEPC-treated H₂O (RNase free) and the RNA concentration is determined by measuring the OD₂₆₀ in a SpectraMax photometer (Molecular Devices, Sunnyvale, Calif.).

Sequences for primers (BIG, Freiburg, Germany) and TaqMan probes for Q-PCR containing 5′ FAM fluorescent reporter and 3′ TAMRA quencher dyes (Eurogentec, Seraing, Belgium) are selected using the Primer Express Software (Applied Biosystems, Foster City, Calif.) at default settings; Primer TM Requirements: Min Tm: 58° C., Max Tm:60° C., Optimal Tm: 59° C., Maximal Tm difference: 2° C. Primer GC Content Requirements: Min %GC 30, Max %GC 80, 3′ clamp of 0 residues. Primer Length Requirements: Min length: 9, Max length: 40, optimal length: 20. Amplicon Requirements: Min Tm: 0° C., Max Tm: 85° C., Min length: 50, Max length: 150. TaqMan Probe Criteria: TaqMan probe Tm must be 10° C. greater than PCR primer Tm.

Primers used are:

| | | |
|---|---|---|
| Forward: | 5′-CAACCCTCCTCGCTTTGTGA-3′; | Seq. ID. No 1 |
| Reverse: | 5′-GACATTGGCTAGGGTGGCAT-3′; | Seq. ID. No 2 |
| Taqman: | 5′-CGCCTGAGCCCTACTCCCTGCA-3′. | Seq. ID. No 3 |

Q-PCR is performed using the TaqMan PCR Core reagent kit (Applied Biosystems Foster City, Calif.) in a ABI Prism 7700 sequence detector (Applied Biosystems) according to the manufacturers instructions. 50 ng total RNA in 12 μl DEPC-H₂O are prepared in MicroAmp optical 96 well reaction plates (Applied Biosystems) together with 13 μl TaqMan PCR reagent mix as provided in Table 1.

TABLE 1

| Reagent | Volume/sample | Final concentration |
|---|---|---|
| 10 × TaqMan buffer | 2.5 μl | 1x |
| 25 mM MgCl₂ | 5.5 μl | 5.5 mM |
| 10 mM dATP | 0.75 μl | 300 μM |
| 10 mM dCTP | 0.75 μl | 300 μM |
| 10 mM dGTP | 0.75 μl | 300 μM |
| 20 mM dUTP | 0.75 μl | 600 μM |
| RNase inhibitor (20 u/μl) | 0.25 μl | 0.2 u/μl |
| AmpliTaq GOLD (5 u/ml) | 0.125 μl | 0.025 u/μl |
| MuLV rev.transcript.(50 u/ml) | 0.125 μl | 0.25 u/μl |
| 10 μM forward primer | 0.5 μl | 200 nM |
| 10 μM reverse primer | 0.5 μl | 200 nM |
| 5 μM TaqMan probe | 0.5 μl | 100 nM |
| 50 ng RNA in DEPC-H₂O | 12 μl | 2 ng/μl |

Settings for reverse transcription of the target RNA and detection by Q-PCR are 50° C. for 2 min., 95° C. for 10 min. followed by 50 cycles of 95° C. for 15 sec and 60° C. for 1 min. Relative measurement of the amplified product is performed using the comparative C_{T} method as described in the manufacturer's manual (Applied Biosystems, ABI Prism 7700 sequence detection system, User Bulletin #2).

Data using real time Q-PCR indicate that Fra-1 is highly repressed by treatment with the HDAC inhibitor as seen in both H1299 cells (Tables 2, 4) and A549 cells (Tables 3, 5) over a wide dose range 10-1000 nM. These data also confirm results obtained using conventional microarray technology which indicate that the expression of Fra-1 mRNA is highly repressed (>200 fold) in the same cells in the presence of TPX A (data not shown).

TABLE 2
Relative levels of Fra-1 mRNA in H1299 cells
after 24 hours Tpx A treatment measured by Q-PCR

| Concentration of TPX | % Fra1 mRNA |
|---|---|
| A (nM) | remaining |
| 0 | 100 |
| 1 | 98 |
| 10 | 2 |
| 50 | 1 |
| 100 | 1 |
| 250 | 1 |
| 500 | 1 |
| 1000 | 1 |
| DMSO 10 | not determined |
| DMSO 1000 | not determined |

TABLE 3
Relative levels of Fra-1 mRNA in A549 cells
after 24 hours Tpx A treatment measured by Q-PCR.

| Concentration of TPX | % Fra1 mRNA |
|---|---|
| A (nM) | remaining |
| 0 | 103 |
| 1 | 95 |
| 10 | 64 |
| 50 | 26 |
| 100 | 16 |
| 250 | 6 |
| 500 | 4 |
| 1000 | 3 |
| DMSO 10 | 106 |
| DMSO 1000 | 88 |

TABLE 4
Relative levels of Fra-1 mRNA in H1299 cells
after 48 hours Tpx A treatment measured by Q-PCR

| Concentration of TPX | % Fra1 mRNA |
|---|---|
| A (nM) | remaining |
| 0 | 101 |
| 1 | 94 |
| 10 | 7 |
| 50 | 1 |
| 100 | 1 |
| 250 | 1 |
| 500 | 1 |
| 1000 | 1 |
| DMSO 10 | 82 |
| DMSO 1000 | 78 |

TABLE 5
Relative levels of Fra-1 mRNA in A549 cells
after 48 hours Tpx A treatment measured by Q-PCR.

| Concentration of TPX | % Fra1 mRNA |
|---|---|
| A (nM) | remaining |
| 0 | 104 |
| 1 | 93 |
| 10 | 84 |
| 50 | 38 |
| 100 | 32 |
| 250 | 14 |
| 500 | 7 |
| 1000 | 6 |
| DMSO 10 | 106 |
| DMSO 1000 | 77 |

The IC50's determined as ranging between 1-10 nM and 10-50 nM in the H1299 cell line and in the A549 cell line, respectively.

### Example 2

### Differential Expression of Fra-1 in H1299 and HCT116 Cells After Treatment with Tpx A or Compound A

H1299 cells and HCT116 cells (ATCC, Manassas, Va.) are cultured and exposed to Tpx A or Compound A and levels of Fra-1 message assayed. In these experiments, H1299 cells are cultured as described in Example 1. HCT116 cells are cultured in RPMI 1640 medium containing 10% fetal calf serum at 37° C. and 5% CO₂. 100 nM trapoxin, 100 nM Compound A or 100 nM DMSO are added to the cells at 60% confluence and grown up for either 6 or 24 hours. Using methodologies as described in Example 1 above, the RNA from cultures treated with Tpx A, Compound A or DMSO as a control is obtained and analysed by Q-PCR for Fra-1 levels; in this case, 8.7 ng total RNA in 12 μl DEPC-H₂O are prepared in MicroAmp optical 96 well reaction plates (Applied Biosystems) together with 13 μl TaqMan PCR reagent mix as provided in Table 1.

Data in Tables 6 and 7 indicate that Fra-1 message is down regulated compared to control cultures not only in H1299 and HCT116 cells treated with Tpx A but also in cultures treated with Compound A.

TABLE 6
Relative levels of Fra-1 mRNA in H1299 cells after treatment with HDAC
inhibitors measured by Q-PCR at 2 timepoints.

| | % Fra1 mRNA |
|---|---|
| Drug added | remaining |
| DMSO 6 hr | 100.96 |
| DMSO 24 hr | 94.83 |
| TPX A 6 hr | 5.81 |
| TPX A 24 hr | 3.11 |
| Compound A 6 hr | 7.14 |
| Compound A 24 hr | 2.07 |

TABLE 7
Relative levels of Fra-1 mRNA in HCT116 cells after treatment with
HDAC inhibitors measured by Q-PCR at 2 timepoints.

| | % Fra1 mRNA |
|---|---|
| Drug added | remaining |
| DMSO 6 hr | 100.48 |
| DMSO 24 hr | 56.17 |
| TPX A 6 hr | 14.25 |
| TPX A 24 hr | 13.68 |
| Compound A 6 hr | 32.59 |
| Compound A 24 hr | 15.89 |

Additional analysis of H1299 and HCT116 cells treated with other HDAC inhibitors (data not shown) also indicates reduced levels of Fra-1 expression in the presence of these inhibitors, thus enforcing the link between Fra-1 levels and HDAC activity. The fact that different HDAC inhibitors can cause inhibition of Fra1 expression in different cell lines is strong evidence that the down regulation of Fra-1 message level is a result of HDAC inhibition and not due to unrelated side effects associated with treatment of particular cells with a particular compound.

### Example 3

### Differential Expression of Fra-1 in A549 Cells After Treatment with Compound B

Human A549 non-small cell lung carcinoma cells (ATCC, Manassas, Va.) (ATCC #CCL-185) are cultured in DMEM medium (Life Technologies, Gaithersburg, Md.) containing 10% fetal bovine serum (FBS, Life Technologies) plus 50 μg/ml Gentamycin (Life Technologies) in a humidified incubator with a 5% CO₂ atmosphere at 37° C. Prior to Compound B treatment, A549 cells are plated at a density of 5×10⁴ cells/well in 24 well plates (Costar, Corning, N.Y.) and grown up for 24 h. Compound B (10 mM stock solution in DMSO) is diluted to a final concentration of 100 nM in tissue culture medium and added to the cells. As control an equivalent amount of DMSO is added to control cultures without Compound B. The cells are incubated at 37° C. for either 16, 24 or 48 hrs. All cell cultures are set up in duplicate. Total RNA from the compound—treated as well as untreated A549 cells is prepared using the RNeasy Mini kit (Qiagen, Hilden, Germany) according to the manufacturer's instructions. The RNA is recovered in 50 μl DEPC-treated H₂O (RNase free) and the RNA concentration is determined by measuring the OD₂₆₀ in a SpectraMax photometer (Molecular Devices, Sunnyvale, Calif.). The amount of RNA is adjusted to 5 ng/microliter. For each RNA sample duplicate 25 microliter Q-PCR reactions are setup as listed in Table 8.

TABLE 8

| Reagent | Volume/sample | Final concentration |
|---|---|---|
| 10 × TaqMan buffer | 2.5 μl | 1x |
| 25 mM MgCl₂ | 5.5 μl | 5.5 mM |
| 10 mM dATP | 0.75 μl | 300 μM |
| 10 mM dCTP | 0.75 μl | 300 μM |
| 10 mM dGTP | 0.75 μl | 300 μM |
| 20 mM dUTP | 0.75 μl | 600 μM |
| RNase inhibitor (20 u/μl) | 0.25 μl | 0.2 u/μl |
| AmpliTaq GOLD (5 u/ml) | 0.125 μl | 0.025 u/μl |
| MuLV rev.transcript.(50 u/ml) | 0.125 μl | 0.25 u/μl |
| 10 μM forward primer | 0.5 μl | 200 nM |
| 10 μM reverse primer | 0.5 μl | 200 nM |
| 5 μM TaqMan probe | 0.5 μl | 100 nM |
| 50 ng RNA in DEPC-H₂O | 10 μl | 2 ng/μl |
| DEPC-H₂O | 2 μl | |

DNA sequence of the Fra-1 primers and probes are as described above. Settings for reverse transcription of the target RNA and detection by Q-PCR are 50° C. for 2 min., 95° C. for 10 min. followed by 50 cycles of 95° C. for 15 sec and 60° C. for 1 min. Relative measurement of the amplified product is performed using the comparative C_{T} method as described in the manufacturer's manual (Applied Biosystems, ABI Prism 7700 sequence detection system, User Bulletin #2). Results are expressed as a percentage of the amount of Fra-1 mRNA recovered from untreated cells. Data indicate that Fra-1 is highly repressed over a prolonged period of time in A549 cells exposed to Compound B compared to controls (Table 9).

TABLE 9
Relative levels of Fra-1 mRNA in A549 cells after treatment with HDAC
inhibitor Compound B measured by Q-PCR at 3 timepoints.

| Drug added | % Fra1 mRNA remaining | STDEV (n = 4) |
|---|---|---|
| DMSO 16 hr | 100.0 | 14.6 |
| DMSO 24 hr | 100.0 | 0.9 |
| DMSO 48 hr | 100.0 | 0.7 |
| Compound B 16 hr | 15.0 | 1.5 |
| Compound B 24 hr | 14.1 | 0.6 |
| Compound B 48 hr | 52.8 | 4.7 |

### Example 4

### Histone Deactylase Activity in the presence of Trapoxin A, Compound A and Compound B

Using conventional methodologies, the actual enzyme activity of Histone H4 was examined in the presence of Trapoxin A, Compound A or Compound B. Briefly, 30,000 cpms of a tritium labeled acetylated Histone H4 peptide is incubated with histone deacetylase activity purified from H1299 cells by ion exchange chromatography in HDAC assay buffer (10 mM Tris-Cl, pH 8.0, 10 mM NaCl, 10% glycerol) in the presence or absence of drug. The mixture is allowed to sit for 2 hours at 37° C. and radio-labeled acetyl groups released by histone deacetylase activity extracted into ethyl acetate and counted on a scintillation counter. The IC50 represents the concentration of drug that inhibits 50% of the histone deacetylase activity obtained in the absence of drug. Data verify that Trapoxin A, Compound A and Compound B possess HDAC inhibitory activity and support the finding that administration of these compounds to cells as disclosed in the Examples contained herein not only results in the down regulation of Fra-1 but that this down regulation is associated with actual HDAC inhibition in these cells (see Table 10).

TABLE 10
HDAC activity in the presence of Trapoxin, Compound A and
Compound B

| Compound | IC50/μM on histone deacetylase |
|---|---|
| Trapoxin | 2.2 ± 0.96 |
| Compound A | 0.063 ± 0.01 |
| Compound B | 0.032 ± 0.017 |

1. A method for screening for HDAC inhibition in cells in vitro comprising
a). detecting Fra-1 levels in said cells and in control cells; and,
b). comparing Fra-1 levels in said cells and in said control cells
wherein HDAC inhibition is associated with Fra-1 level down regulation and wherein similar or up regulated levels of Fra-1 compared to control levels indicate that HDACs are not inhibited in said cells in vitro.

2. The method of claim 1 wherein said Fra-1 levels is the level of Fra-1 mRNA.

3. The method of claim 1 wherein said Fra-1 levels is the level of Fra-1 protein.

4. A method for screening for HDAC inhibition in a subject in vivo comprising
a). detecting Fra-1 levels in said subject in vivo and in a control subject; and,
b). comparing Fra-1 levels in said subject in vivo and in said control subject
wherein HDAC inhibition is associated with Fra-1 level down regulation and wherein similar or up regulated levels of Fra-1 compared to control levels indicate that HDACs are not inhibited in said subject in vivo.

5. The method of claim 4 wherein said Fra-1 levels is the level of Fra-1 mRNA.

6. The method of claim 4 wherein said Fra-1 levels is the level of Fra-1 protein.

7. A method for screening a compound for HDAC inhibitory activity in vitro, comprising
a). administering a compound to cells in vitro;
b). assaying for Fra-1 levels in said cells and in control cells to which no compound has been administered; and,
c) comparing levels of Fra-1 in said cells and in said control cells
wherein HDAC inhibition is associated with Fra-1 level down regulation and wherein similar or up regulated levels of Fra-1 in said cells compared to Fra-1 levels in control cells indicate that said compound does not have HDAC inhibitory activity.

8. The method of claim 7 wherein said Fra-1 levels is the level of Fra-1 mRNA.

9. The method of claim 7 wherein said Fra-1 levels is the level of Fra-1 protein.

10. A method for screening a compound for HDAC inhibitory activity in a subject in vivo, comprising
a). assaying Fra-1 levels in said subject;
b). administering a compound to said subject;
c). reassaying Fra-1 levels in said subject after administration of the compound; and,
d) comparing Fra-1 levels in said subject before and after administration of the compound
wherein HDAC inhibition is associated with Fra-1 level down regulation and wherein similar or up regulated levels of Fra-1 in said subject after administration of the compound compared to levels before compound administration indicate that said compound does not have HDAC inhibitory activity.

11. The method of claim 10 wherein said Fra-1 levels is the level of Fra-1 mRNA.

12. The method of claim 4 wherein said Fra-1 levels is the level of Fra-1 protein.

13. A method for monitoring the therapeutic efficacy of a known HDAC inhibitor in a subject comprising
a). detecting Fra-1 levels in said subject before and after treatment with said HDAC inhibitor; and,
b). comparing Fra-1 levels in said subject
wherein HDAC inhibition is associated with Fra-1 level down regulation and wherein down regulation of Fra-1 levels in said subject after treatment compared to levels before treatment indicate that the HDAC inhibitor is therapeutically effective.

14. The method of claim 13 wherein said Fra-1 levels is the level of Fra-1 mRNA.

15. The method of claim 13 wherein said Fra-1 levels is the level of Fra-1 protein.

16. A method to determine resistance of a cell to a known HDAC inhibitor comprising
a). administering said HDAC inhibitor to said cells in vitro; and,
b). screening for Fra-1 levels in said cells and in control cells to which no HDAC inhibitor has been administered; and,
c). comparing levels of Fra-1 in said cells and in said control cells
wherein HDAC inhibition is associated with Fra-1 level down regulation and wherein a lack of Fra-1 level down regulation in said cells compared to Fra-1 levels in the control cells indicates resistance of said cells to the HDAC inhibitor.

17. The method of claim 16 wherein said Fra-1 levels is the level of Fra-1 mRNA.

18. The method of claim 16 wherein said Fra-1 level is the level of Fra-1 protein.

19. A method to determine resistance of a subject to a known HDAC inhibitor comprising
a). detecting Fra-1 levels in said subject before and after treatment with said HDAC inhibitor; and,
b). comparing Fra-1 levels in said subject
wherein HDAC inhibition is associated with Fra-1 level down regulation and wherein a lack of Fra-1 level down regulation in said subject after treatment compared to Fra-1 levels before treatment indicates resistance of said subject to the HDAC inhibitor.

20. The method of claim 19 wherein said Fra-1 levels is the level of Fra-1 mRNA.

21. The method of claim 19 wherein said Fra-1 level is the level of Fra-1 protein.

22. A method of diagnosing a proliferative disease susceptible to treatment with HDAC inhibitory agents, which comprises measuring in cells of the subject which exhibits the proliferative disease a decreased level of Fra-1 mRNA or Fra-1 protein.

23. The method of claim 22 wherein the measurement takes place ex vivo.

24. Use of Fra-1 as a biomarker for HDAC inhibitory activity.
